(19) United States
(12) Patent Application Publication
(10) Pub. No.: US 2025/0308069 A1
(43) Pub. Date: Oct. 2, 2025

(54) CALIBRATING A DIGITAL IMAGE OF SKIN TISSUE

(71) Applicant: AI LABS GROUP, S.L., Bilbao (Bizkaia) (ES)

(72) Inventors: Taig MAC CARTHY ESPINAR, Bilbao (Bizkaia) (ES); Alfonso MEDELA AYARZAGUENA, Bilbao (Bizkaia) (ES)

(21) Appl. No.: 18/866,642

(22) PCT Filed: Apr. 13, 2023
(86) PCT No.: PCT/EP2023/059688

§ 371 Date: Nov. 18, 2024

(30) Foreign Application Priority Data
May 25, 2022 (EP) 22382500.1

(51) Int. Cl. G06T 7/80; G06T 7/90

(52) U.S. Cl. CPC G06T 7/80; G06T 7/90; G06T 2207/20132; G06T 2207/30088; G06T 2207/30204

(57) ABSTRACT

A computer-implemented method is for calibrating a digital image of skin tissue. The digital image includes a calibration marker located on or near the skin tissue. The calibration marker has benchmark elements wherein at least one benchmark attribute value of at least one attribute type. The computer-implemented method detects the calibration marker within the digital image; and detects the benchmark elements within a cropped portion of the digital image that includes the calibration marker. The computer-implemented method further includes, for the respective benchmark elements, determining at least one depicted attribute value of the at least one attribute type based on pixels of the digital image located within the respective benchmark elements; and calibrating the digital image by correcting deviations between the depicted attribute values and the benchmark attribute values associated with the respective benchmark elements.

### FIELD OF THE INVENTION

The present invention generally relates to calibrating digital images, in particular digital images of skin tissue.

### BACKGROUND OF THE INVENTION

Several skin conditions, e.g. psoriasis and dermatitis, require assessment of skin lesions to determine the severity and evolution of the skin condition. This assessment is typically performed by an expert during a time-consuming manual process that is prone to inter-expert variability. As such, software applications are being developed to at least partially automate this assessment based on digital images of the skin lesion captured by the patient, e.g. by means of a smartphone. This has the problem that visible signs indicative for the severity and evolution of skin conditions, e.g. lesion colour and size, can be substantially distorted in a digital image thereby resulting in a sub-optimal assessment of the skin condition.

### SUMMARY OF THE INVENTION

It is an object of the present invention, amongst others, to solve or alleviate the above identified problems and challenges by calibrating digital images of skin tissue.

According to a first aspect, this object is achieved by a computer-implemented method for calibrating a digital image of skin tissue, wherein the digital image comprises a calibration marker located on or near the skin tissue. The calibration marker comprises benchmark elements characterised by at least one benchmark attribute value of at least one attribute type. The computer-implemented method comprises:
detecting the calibration marker within the digital image;
detecting the benchmark elements within a cropped portion of the digital image that includes the calibration marker;
for the respective benchmark elements, determining at least one depicted attribute value of the at least one attribute type based on pixels of the digital image located within the respective benchmark elements; and
calibrating the digital image by correcting deviations between the depicted attribute values and the benchmark attribute values associated with the respective benchmark elements.

The calibration marker can be included in the digital image by placing the calibration marker on or near the skin tissue upon capturing a digital image of the skin tissue. The calibration marker may be a paper strip or plastic film upon which one or more benchmark elements are provided. The calibration marker may, for example, be a transparent plastic film upon which a plurality of benchmark elements are printed.

Detecting the calibration marker within the digital image allows obtaining a cropped portion of the digital image that includes the calibration marker. In other words, a portion of the digital image that includes the calibration marker can be identified. The cropped portion allows detecting the benchmark elements faster and more efficiently, as the portion of the digital image that excludes the cropped portion is omitted from the detecting of benchmark elements. In other words, the search space for detecting the one or more benchmark elements is substantially reduced.

The respective benchmark elements are characterized by one or more benchmark attribute values of one or more attribute types. In other words, a single benchmark element can be characterized by a plurality of benchmark attribute values associated with respective attribute types. An attribute type may, for example, be a colour, a location, a dimension, a surface area, a pixel density, an orientation, or a shape. A benchmark attribute value refers to a known standard or point of reference of a certain attribute type. A depicted attribute value refers to a value of a certain attribute type as represented or rendered in the digital image. Comparing depicted attribute values with benchmark attribute values allows determining deviations between the appearance of skin tissue as represented or rendered in the digital image and the appearance of skin tissue in reality. By correcting these deviations, a calibrated or normalized digital image is obtained wherein the skin tissue is rendered substantially true, i.e. the skin tissue is represented within the digital image as an observer would perceive the skin tissue in reality. The one or more benchmark elements of the calibration marker thus serve as references for calibrating the digital image.

Calibrating the digital image results in a calibrated representation of visible signs, e.g. lesion colour and size, indicative for the severity and the evolution of skin conditions. This has the advantage that a skin condition can be assessed more accurately, reliably, and objectively based on a digital image of skin tissue. The calibrated digital image of skin tissue has the further advantage that it can be used to train a machine learning model, or as an input to a machine learning model for determining the severity and/or evolution of a skin condition.

According to an embodiment, detecting the calibration marker may be performed by a machine learning model trained for detecting the calibration marker within a digital image.

Detecting the calibration marker may, for example, be achieved by means of object detection or object recognition. The machine learning model may be obtained by training a classifier based on annotated digital images of skin tissue that include the calibration marker. Herein, the calibration marker may be labelled. The machine learning model may for example be based on, amongst others, a neural network, a support vector machine, or a convolutional neural network. Alternatively, the machine learning model may be obtained by unsupervised learning or by reinforcement learning.

According to an embodiment, detecting the benchmark elements may be performed by a machine learning model trained for assigning respective pixels within the cropped portion of the digital image to the benchmark elements.

The machine learning model may thus be configured to classify the individual pixels within the cropped portion of the digital image to respective classes associated with the benchmark elements, e.g. by semantic image segmentation. In doing so, a pixel map or mask can be generated that allows to identify the pixels that are part of the respective benchmark elements, thereby detecting the benchmark elements.

According to an embodiment, one or more benchmark elements may be characterised by respective benchmark colour values of a colour type; and wherein calibrating the digital image comprises correcting colour values of pixels within the digital image.

A colour value of the colour type can be indicative for, amongst others, a hue, a brightness, a saturation, and/or a luminance. The colour value may be a value according to any colour model such as, for example, an RGB colour model, a CMYK colour model, a YUV colour model, an HSL colour model, and an HSV colour model. One or more benchmark elements within the calibration marker may thus be characterized by a predetermined colour value, i.e. a benchmark colour value. Preferably, the calibration marker comprises a plurality of benchmark elements characterised by respective benchmark colour values. Preferably, the respective benchmark colour values include skin tones such as, for example, [0, 20, 10, 0] CMYK, [0, 20, 30, 0] CMYK, [0, 30, 30, 50] CMYK, and [0, 40, 40, 50] CMYK.

This allows comparing the colour value of a benchmark element as depicted in the digital image with the benchmark colour value, thereby obtaining a deviation in the colour value. This colour deviation can be substantially corrected to calibrate the digital image. Correcting colour values may include adjusting the colour value of the pixels within the digital image such that the respective colour deviations associated with the different benchmark colour values are minimized. In other words, the colour deviation associated with a benchmark colour value is minimized to an extent that the adjusting does not substantially worsen or increases the colour deviation associated with any other benchmark colour values. Thus, correcting the colour values of the digital image is a multi-variable optimization with the objective to minimize the colour deviation associated with the respective benchmark elements. This avoids that correcting a colour deviation for a certain colour value, e.g. red, generates additional deviations or colour-shifts for other colour values, e.g. blue and green. This can for example be achieved by an optimization method based on partial least squares regression.

Calibrating the colour values allows assessing skin tissue more reliably based on a digital image regardless of factors that influence the colours of a digital image, e.g. ambient lighting and chromatic distortion. This has the advantage that the colour of skin lesions may be rendered substantially true within the digital image regardless of the conditions wherein the digital image is taken or which equipment has been used to capture the digital image. This has the further advantage that the severity and/or evolution of a skin condition can be determined more reliably based on a digital image.

According to an embodiment, determining at least one depicted attribute value of the colour type comprises determining an average depicted colour value of the pixels of the digital image located within the respective one or more benchmark elements.

The average depicted colour value may thus be obtained by determining the depicted colour value for each pixel within a respective benchmark element, and determining the average of these depicted colour values.

According to an embodiment, the computer-implemented method may further comprise determining respective colour deviations between the average depicted colour value and the benchmark colour value associated with the respective one or more benchmark elements.

According to an embodiment, at least four benchmark elements may be characterised by a benchmark location value of a location type; and calibrating the digital image may comprise adjusting a perspective of the digital image.

At least four benchmark elements within the calibration marker may thus have a predetermined location on the calibration marker. The respective benchmark location values may be indicative for these predetermined locations. The respective benchmark location values may, for example, be coordinates according to a coordinate system having a first axis along a longitudinal edge of the calibration marker and a second axis along a transverse edge of the calibration marker.

Adjusting the perspective of the digital image allows assessing skin tissue more reliably based on a digital image regardless of factors that influence the perspective of a digital image, e.g. optical aberrations or camera angle. This has the advantage that the size and shape of skin lesions may be rendered substantially true within the digital image regardless of the conditions wherein the digital image is taken or which equipment has been used to capture the digital image. This has the further advantage that the severity and/or evolution of a skin condition can be determined more reliably, as the shape and size of a skin lesion are typical visible signs for skin conditions.

According to an embodiment, determining at least one depicted attribute value of the location type comprises determining a depicted location of the respective at least four benchmark elements.

Any pixel or point within the respective at least four benchmark elements characterised by a benchmark location value may be indicative for the depicted location of the benchmark element. For example, a pixel at the centre of a respective benchmark element can be indicative for the location of the benchmark element. Determining the depicted location thus includes determining the location of said pixel.

According to an embodiment, the computer-implemented method may further comprise determining a depicted polygon defined by the depicted locations of the respective at least four benchmark elements within the digital image; and wherein adjusting the perspective of the digital image further comprises mapping pixels within the depicted polygon to pixels within a benchmark polygon defined by the benchmark location value of the at least four benchmark elements.

The at least four benchmark elements characterized by a benchmark location value may thus define the benchmark polygon. In other words, the respective corners of the benchmark polygon may be defined by the respective benchmark location values of the at least four benchmark elements. Similarly, the respective corners of the depicted polygon may be defined by the respective depicted locations of the at least four benchmark elements. The perspective of the digital image may be adjusted based on the benchmark polygon and the depicted polygon. This can be achieved by a geometric image transformation that warps the depicted polygon to the shape of the benchmark polygon, i.e. the pixel grid of the digital image is deformed based on the deviation between the depicted polygon and the benchmark polygon.

According to an embodiment, one or more benchmark elements may define a reference axis of the calibration marker, and wherein calibrating the digital image comprises rotating the digital image based on the reference axis.

The calibration marker may comprise a plurality of benchmark elements that are lined up, thereby defining the reference axis. The reference axis may, for example, be defined by respective predetermined pixels or points within the plurality of lined-up benchmark elements, e.g. the geometric centre of the respective benchmark elements. The plurality of benchmark elements may further be characterized by one or more additional attribute values of different attribute types, e.g. colour values. This has the further advantage that the digital image can be rotated based on the reference axis without providing additional benchmark elements on the surface area of the calibration marker. Alternatively, the calibration marker may comprise a benchmark element substantially shaped as a line or an arrow indicative for the reference axis.

According to an embodiment, the computer-implemented method may further comprise determining an angle between the reference axis of the calibration marker and a predetermined edge of the digital image.

The predetermined edge 513 can be any outer edge of the digital image 510. This allows rotating the digital image to a calibrated position by rotating the digital image such that the references axis is oriented substantially horizontal or vertical. To this end, the digital image can be rotated by an angle corresponding to the determined angle between the reference axis and the predetermined edge.

According to an embodiment, the reference axis may be characterised by a benchmark location on the calibration marker; and wherein rotating the digital image may further be based on a depicted location of the reference axis.

The reference axis may be arranged on the surface of the calibration marker such that the calibration marker is asymmetric. For example, the reference axis may be arranged on the calibration marker with a bias towards an outer limit of the calibration marker. This allows orienting the calibration marker according to a predetermined orientation. This can avoid that the digital image is rotated to an inverted orientation.

According to an embodiment, one or more benchmark elements may be characterized by a benchmark surface area value of a surface area type; the computer-implemented method may further comprise determining a surface area associated with the respective pixels of the digital image based on an amount of pixels located within the respective one or more benchmark elements and the benchmark surface area of the respective one or more benchmark elements.

In other words, one or more benchmark elements may be characterized by a predetermined or known surface area, i.e. the benchmark surface area value. As such, a depicted surface area can be determined for each pixel within these one or more benchmark elements in the digital image. This can, for example, be achieved by dividing the benchmark surface area value for the respective benchmark elements by the amount of pixels within the respective benchmark elements. This allows determining the size or dimensions of skin lesions depicted in the digital image based on the amount of pixels within the skin lesion. This has the further advantage that the severity and/or evolution of a skin condition can be determined more reliably based on a digital image, as the size of a skin lesion is a typical visible sign for skin conditions.

According to an embodiment, determining the surface area associated with pixels located outside the one or more benchmark elements may comprise interpolating and/or extrapolating the surface area associated with the pixels located within the one or more benchmark elements.

In other words, a surface area per pixel may be determined for the pixels that are not located within the one or more benchmark elements characterized by a benchmark surface area. For pixels within the digital image that are located between at least a first and a second benchmark element, this can be achieved by interpolating the surface area associated with the pixels located within the respective benchmark elements. This allows accurately determining the size or dimensions of skin lesions located between the benchmark elements, e.g. inside the boundaries of the calibration marker. For pixels within the digital image that are not located between at least two benchmark elements, e.g. pixels located at a substantial distance from the calibration marker, this can be achieved by extrapolating the surface area associated with the pixels located within the respective benchmark elements. This allows accurately determining the size or dimensions of skin lesions not located between benchmark elements, e.g. outside the boundaries of the calibration marker.

According to a second aspect, the invention relates to a data processing system configured to perform the computer-implemented method according to the first aspect.

According to a third aspect, the invention relates to a computer program comprising instructions which, when the computer program is executed by a computer, cause the computer to perform the computer-implemented method according to the first aspect.

According to a fourth aspect, the invention relates to a computer-readable medium comprising instructions which, when executed by a computer, cause the computer to perform the computer-implemented method according to the first aspect.

### BRIEF DESCRIPTION OF THE DRAWINGS

FIG. 1 shows steps according to a computer-implemented method for calibrating a digital image of skin tissue according to embodiments;

FIG. 2 shows steps of the computer-implemented method wherein calibrating the digital image comprises correcting colour values within the digital image, according to embodiments;

FIG. 3 shows steps of the computer-implemented method wherein calibrating the digital image comprises adjusting a perspective of the digital image, according to embodiments;

FIG. 4 shows steps of the computer-implemented method for determining a surface area associated with the respective pixels of the digital image, according to embodiments;

FIG. 5 shows steps of the computer-implemented method further comprising rotating the digital image based on a reference axis, according to embodiments; and

FIG. 6 shows an example embodiment of a suitable computing system for performing steps according to example aspects of the disclosure.

### DETAILED DESCRIPTION OF EMBODIMENT(S)

Skin conditions such as, for example, psoriasis and dermatitis, are typically assessed using a scoring system or a scoring tool. These scoring systems typically combine an assessment of the severity of skin lesions and an extent of the skin area affected by skin lesions into a single score. A skin lesion refers to any area of skin tissue or portion of skin tissue that has substantially different characteristics from the surrounding skin tissue, e.g. a different colour, shape, size, or texture. Examples of scoring systems are the psoriasis area and severity index, PASI, and scoring atopic dermatitis, SCORAD. Periodically repeating the assessment of a skin condition by means of such a scoring system typically allows determining the evolution of the skin condition in time, and can thus allow determining therapeutic efficacy.

Typically, a scoring system is based on the interpretation of visible signs indicative for the extent or severity of the skin condition, also referred to as visible indicators or clinical signs. Such visible signs typically include, amongst others, erythema or redness, induration or thickness, desquamation or scaling, swelling, effect of scratching, oozing, crust formation, lichenification, and dryness. Additionally, the extent of the affected area contributes to the assessment of the skin condition. In other words, the size or dimensions of the skin lesions are indicative for the severity of the skin condition.

A problem of such scoring systems is that the assessment is subjective and thus perceptive for interobserver variability, i.e. the result may vary with the person that performs the assessment. Moreover, assessing a skin condition based on a scoring system is typically performed by a trained expert and is time-consuming. As such, software applications are being developed to at least partially automate this assessment based on digital images of skin tissue comprising skin lesions. These digital images may preferably be captured by the patient, e.g. by means of a smartphone or tablet. This has the problem that the visible signs indicative for the severity of skin conditions can be substantially distorted in a digital image, thereby resulting in an incorrect assessment of the skin condition. For example, the colours in the digital image can be distorted due to ambient lighting and/or chromatic aberration. The size, dimensions, focus, and/or perspective of the digital image can for example be distorted due to monochromatic aberrations, or by capturing the digital image at an unsuitable camera angle. It can thus be desirable to calibrate or normalize digital images of skin tissue.

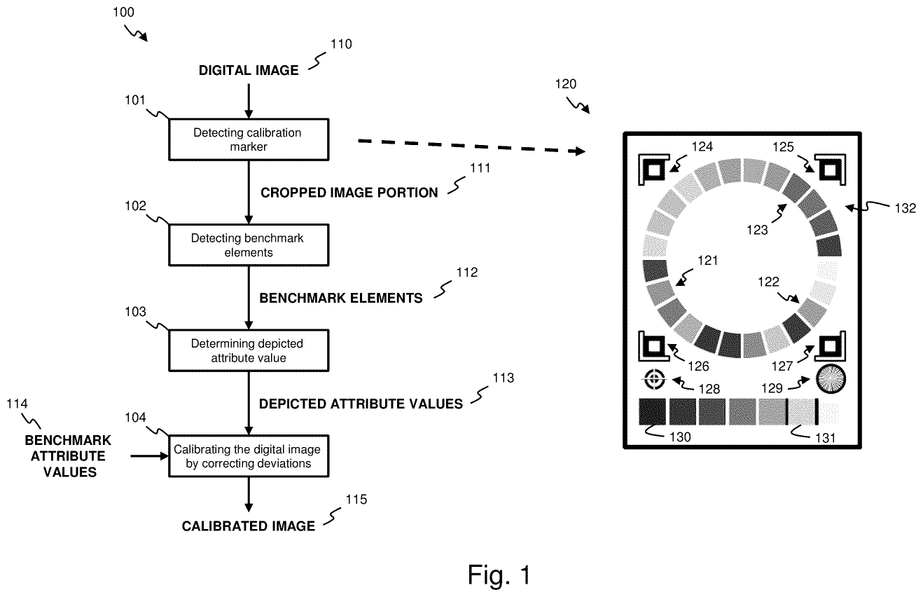

FIG. 1 shows steps 100 according to a computer-implemented method for calibrating a digital image 110 of skin tissue according to embodiments. The digital image 110 may be obtained by means of a camera included in, amongst others, a smartphone, a tablet, a webcam, or a digital reflex camera. The digital image 110 comprises a calibration marker 120 located on or near the skin tissue. The calibration marker 120 can be included in the digital image 110 by placing the calibration marker on or near the skin tissue upon capturing the digital image 110. For example, a person having a skin disorder or skin condition can thus place the calibration marker 120 on or near his skin tissue, e.g. near a skin lesion, and use the camera of his smartphone to obtain the digital image 110.

The calibration marker 120 may be a substantially thin strip or film that comprises one or more benchmark elements 121-131. The calibration marker 120 may, for example, be made of paper or plastic. The one or more benchmark elements may be printed on the calibration marker 120. The calibration marker 120 further comprises an unmarked body 132, i.e. a portion of the calibration marker 120 that is substantially free of benchmark elements 121-131. The calibration marker 120 may be made of a transparent material such that skin tissue underneath the unmarked body 132 of the calibration marker 120 remains visible when covered by the calibration marker. The calibration marker 120 may, for example, be made of a transparent plastic film, a laminated polyethylene transparent paper, and/or polypropylene. Alternatively or complementary, a portion of the unmarked body 132 may be substantially free of material such that skin tissue within this portion remains visible when covered by the calibration marker.

The respective benchmark elements 121-131 are characterized by one or more benchmark attribute values of one or more attribute types. In other words, a single benchmark element 121-131 may be characterized by a plurality of benchmark attribute values of different attribute types. An attribute type may, for example, be a colour, a location, a dimension, a surface area, a pixel density, an orientation, or a shape. A single benchmark element 121-131 may thus, for example, be characterized by a colour value, a location value, and a surface area. A benchmark attribute value 114 refers to a known standard or point of reference of a certain attribute type, i.e. a fixed predetermined value. The respective benchmark elements 121-131 may thus for example be characterized by, amongst others, a known colour value, a known location, a known dimension, or a known surface area.

In a first step 101, the calibration marker 120 is detected within the digital image 110. In doing so, a cropped portion 111 of the digital image 110 that includes the calibration marker 120 is obtained. It will be apparent that the cropped portion 111 merely refers to an identified subsection or sub portion within the digital image 110 that includes at least the calibration marker 120 which may, but must not, include removing the other portion of the digital image 110 that excludes the calibration marker 120.

Detecting the calibration marker 120 from the digital image 110 in step 101 may be performed by a machine learning model trained for detecting the calibration marker 120 within a digital image. This may, for example, be achieved by means of object detection or object recognition. A trained machine learning model may be obtained by supervised learning wherein a training dataset is provided to a classifier. The training dataset may comprise a substantial amount of annotated digital images that include calibration markers. The position of the calibration markers within the respective annotated digital images of the training dataset may thus be labelled or marked. The machine learning model may, for example, be based on a neural network, a support vector machine, or a convolutional neural network. Alternatively or complementary, a trained machine learning model may be obtained by unsupervised learning or by reinforcement learning.

In a next step 102, one or more benchmark elements 121-131 are detected within the cropped portion 111 of the digital image. The cropped portion therefore allows detecting the benchmark elements 121-131 faster and more efficiently, as the portion of the digital image 110 that excludes the cropped portion is omitted from the detecting of benchmark elements. In other words, the search space for detecting the benchmark elements 121-131 is substantially reduced from the entire digital image 110 to the cropped portion 111.

This detecting of the benchmark elements 121-131 may be performed by a machine learning model trained for assigning respective pixels within the cropped portion 111 of the digital image to the respective benchmark elements 121-131. The machine learning model may thus be configured or trained to classify the individual pixels within the cropped portion 111 of the digital image 110 to respective classes associated with the respective benchmark elements 121-131. The machine learning model may further be configured or trained to classify the individual pixels within the cropped portion 111 to a class associated with the unmarked body 132 of the calibration marker 120. This may, for example, be achieved by semantic image segmentation. Herein, a pixel map or mask is generated that assigns or classifies each pixel in a digital image of the calibration marker 120 to a respective class. The pixel map or mask may be generated by manually labelling the individual pixels in a digital image of the calibration marker 120. This allows associating the individual pixels within a digital image to the different benchmark elements 121-131, thereby detecting the benchmark elements.

In a next step 103, at least one depicted attribute value of the at least one attribute type is determined for the respective benchmark elements 121-131 based on the pixels of the digital image 110 located within the benchmark elements 121-131. A depicted attribute value 113 refers to a value of a certain attribute type as represented or rendered in the digital image 110. As described above, attribute values can be substantially distorted in a digital image, e.g. deviating colours or a warped perspective. Comparing the depicted attribute values 114 with the known benchmark attribute values 113 therefore allows determining the extent of these deviations or distortions. As such, the deviation between the appearance of skin tissue as represented or rendered in the digital image 110 and the appearance of skin tissue in reality can be determined.

In a next step 104, the digital image 110 is calibrated by correcting the deviations between the depicted attribute values 114 and the benchmark attribute values 113 associated with the respective benchmark elements 121-131. It will be apparent that only depicted attribute values 114 and benchmark attribute values 113 of the same attribute type can be compared to determine a deviation. By correcting these deviations, a calibrated or normalized digital image 115 is obtained wherein the skin tissue is rendered substantially true, i.e. the skin tissue is represented within the digital image as an observer would perceive the skin tissue in reality. In other words, the representation of the skin tissue in the digital image is objectified by substantially correcting divergences from reality. The one or more benchmark elements 121-131 of the calibration marker 120 thus serve as references for calibrating the digital image 110.

Calibrating the digital image 110 results in a calibrated representation of visible signs, e.g. lesion colour and size, indicative for the severity and the evolution of skin conditions. This has the advantage that a skin condition can be assessed more accurately, reliably, and objectively based on a digital image of skin tissue. The calibrated digital image of skin tissue has the further advantage that it can be used to train a machine learning model, or as an input to a machine learning model for determining the severity and/or evolution of a skin condition. This has the further advantage that assessment of skin conditions can be performed by the patient himself and in a time-efficient manner.

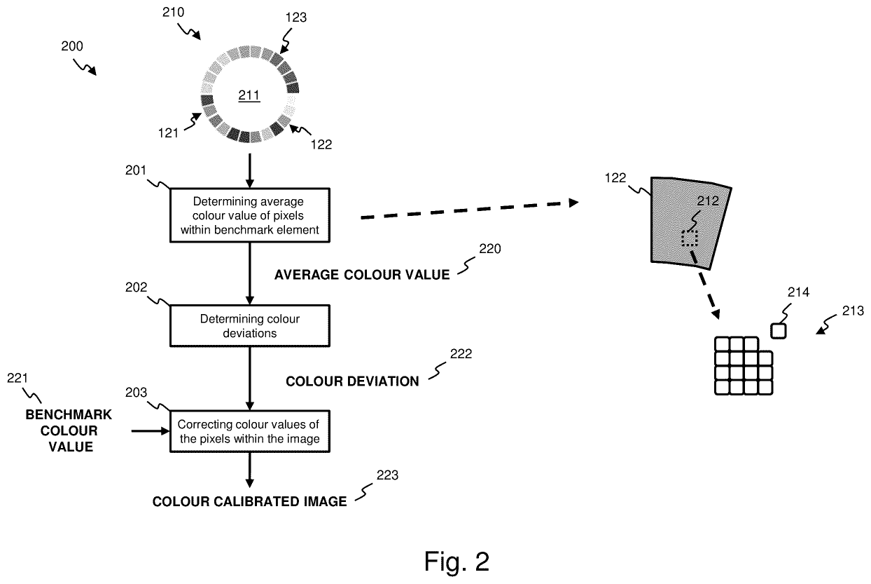

FIG. 2 shows steps 200 of the computer-implemented method wherein calibrating the digital image comprises correcting colour values within the digital image, according to embodiments. One or more benchmark elements 121, 122, 123 within the calibration marker may be characterised by respective benchmark colour values 221 of a colour type. These benchmark elements 121, 122, 123 may be arranged on the calibration marker such that they form a colour roulette 210 or colour wheel, i.e. a circular or ring-shaped arrangement of benchmark elements 121, 122, 123. An inner area 211 enclosed by the ring-shaped arrangement of benchmark elements 121, 122, 123 may be substantially free of benchmark elements, i.e. the inner area 211 may form a portion of the unmarked body of the calibration marker. The inner area 211 may be substantially free of material. In other words, the calibration marker may comprise a cut-out or through-hole that is substantially shaped as the inner area 211. Alternatively, the benchmark elements 121, 122, 123 may be arranged in any another shape that provides an inner area substantially free of benchmark elements, e.g. a rectangular arrangement, an ellipsoid arrangement, or a stadium arrangement.

A colour value of the benchmark elements 121, 122, 123 may be indicative for, amongst others, a hue, a brightness, a saturation, and/or a luminance. The colour value may be a value according to any colour model such as, for example, an RGB colour model, a CMYK colour model, a YUV colour model, an HSL colour model, and an HSV colour model. Benchmark elements 121, 122, 123 may thus be characterized by a predetermined or known colour value, i.e. a benchmark colour value 221.

The respective benchmark colour values 221 of the benchmark elements 121, 122, 123 may include skin tones or skin-like colours that match different skin phototypes. For example, the respective benchmark colour values 221 may correspond to steps on the Fitzpatrick scale. This allows calibrating the digital image more accurately for skin tissue. The respective benchmark colour values 221 may further include non skin-like colours such as, for example, pure pigment values. The calibration marker may, for example, comprise twelve benchmark elements 121, 122, 123 characterized by following respective benchmark colour values according to the CMYK colour model: [0, 20, 10, 0]; [0, 30, 20, 0]; [0, 30, 30, 0]; [0, 20, 30, 0]; [0, 20, 30, 10]; [0, 30, 20, 20]; [0, 20, 30, 20]; [0, 20, 30, 30]; [0, 30, 30, 50]; [0, 20, 30, 50]; [0, 40, 40, 50]; and [0, 40, 40, 70].

Steps 200 may be performed after detecting the benchmark elements in step 102 in FIG. 1. In a first step 201, a depicted colour value may be determined for the respective benchmark elements 121, 122, 123 that are characterized by a benchmark colour value 221. Determining the depicted colour value of a benchmark element 122, i.e. the depicted attribute value of the colour type, may include determining an average depicted colour value 220 of the benchmark element 122. This can be achieved by determining the colour value of the respective pixels 214 within the benchmark element 122 and determining the average colour value 220 for all pixels 213 included in the benchmark element 122. The average may be, amongst others, an arithmetic mean, a median, or a mode.

In a next step 202, the average depicted colour value 220 for each benchmark element 121, 122, 123 characterized by a benchmark colour value 221 may be compared to the benchmark colour value 221 associated with the respective benchmark elements. For example, the average depicted colour value 220 of benchmark element 121 can be compared to the benchmark colour value 221 associated with that benchmark element 121. This allows determining a colour deviation 222 for each benchmark element 121, 122, 123 characterized by a benchmark colour value 221. In other words, a deviation of the digital image from each benchmark colour value 221 can be determined.

In step 203, these colour deviations 222 can be substantially corrected to calibrate the colours of the digital image. Correcting colour values may include adjusting the colour value of the pixels within the digital image such that the respective colour deviations 222 associated with the different benchmark colour values 221 are minimized. In other words, the colour deviation 222 associated with a benchmark colour value 221 is minimized to an extent that the adjusting does not substantially worsen or increases the colour deviation 222 associated with another benchmark colour value 221. Thus, correcting the colour values of the digital image is a multi-variable optimization with the objective to minimize the colour deviation 222 associated with each benchmark element 121, 122, 123. This avoids that correcting a colour deviation for a certain colour value, e.g. red, generates additional deviations or colour-shifts for other colour values, e.g. blue and green. Determining the colour correction to calibrate the digital image can be achieved by, for example, an optimization method based on partial least squares regression. Alternatively, correcting colour values may include adjusting the colour value of pixels within the digital image such that the colour deviations are substantially nullified.

The colour correcting in step 203 thus results in a colour calibrated digital image 223. This allows assessing skin tissue more reliably based on a digital image regardless of factors that influence the colours of a digital image, e.g. ambient lighting and chromatic distortion. This has the advantage that the colour of skin lesions may be rendered substantially true within the colour calibrated digital image 223 regardless of the conditions wherein the digital image is taken or which equipment has been used to capture the digital image. This has the further advantage that the severity and/or evolution of a skin condition can be determined more reliably based on a digital image.

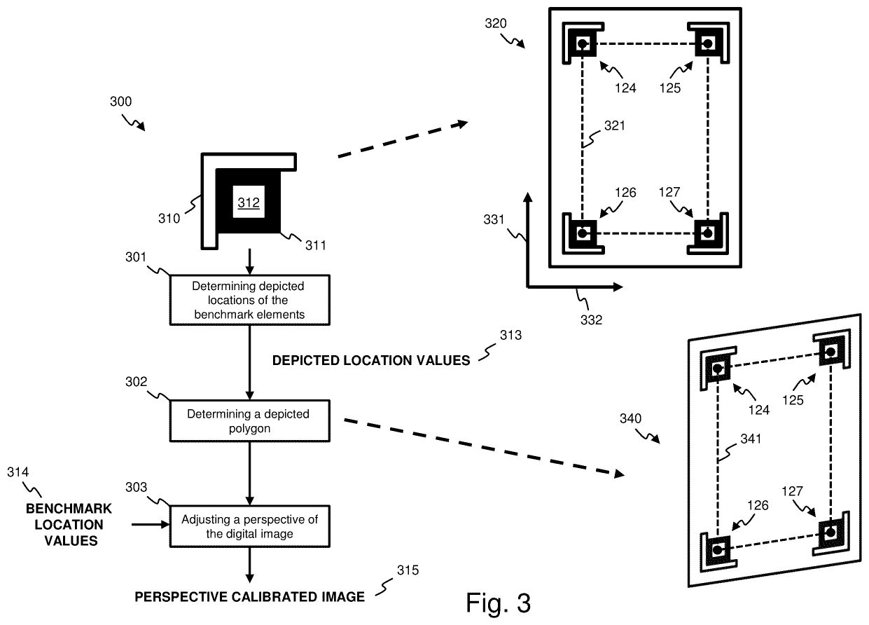

FIG. 3 shows steps 300 of the computer-implemented method wherein calibrating the digital image comprises adjusting a perspective of the digital image, according to embodiments. At least four benchmark elements 124, 125, 126, 127 included in the calibration marker 320 may be characterised by a benchmark location value 314 of a location type. These benchmark elements 124, 125, 126, 127 may thus have a predetermined location on the surface of the calibration marker 320 for which the respective benchmark location values 314 are indicative. The benchmark location values 314 may, for example, be coordinates according to a coordinate system having a first axis 331 along a longitudinal edge of the calibration marker 320 and a second axis 332 along a transverse edge of the calibration marker 320.

The benchmark elements 124, 125, 126, 127 characterised by a benchmark location value 314 may be arranged near an outer limit of the calibration marker, e.g. substantially close to a corner or an edge of the calibration marker 320. The benchmark elements 124, 125, 126, 127 may comprise an outer band 310 that delimits at least part of an inner band 311. The inner band 311 may further enclose an inner area 312. The outer band 310, inner band 311, and inner area 312 may further have substantially contrasting colours, e.g. black and white. This can improve detection of benchmark elements 124, 125, 126, 127.

The benchmark location value 314 of a respective benchmark element 124, 125, 126, 127 may be defined by any point or pixel within the respective benchmark element, e.g. a pixel located at the geometric centre of the benchmark element, a pixel located at the geometric centre of the inner area 312, or a pixel located in a corner of the inner band 311. The benchmark location values 314 associated with the respective benchmark elements 124, 125, 126, 127 may define a benchmark polygon 321. For example, the benchmark location values 314 may define the corners of a rectangle. In other words, the respective benchmark location values 314 may delimit a benchmark polygon 321. The benchmark polygon 321 may be another polygon depending on the amount and arrangement of benchmark elements 124, 125, 126, 127, e.g. a hexagon defined by six benchmark elements. Alternatively or complementary, a benchmark polygon 321 may comprise additional benchmark elements (not shown in FIG. 3) that do not define the corners of the polygon. Such an additional benchmark element may, for example, be located on a leg of the benchmark polygon 321, or within the area enclosed by the polygon 321. For example, benchmark polygon 321 may comprise two additional benchmark elements arranged on the leg between element 124 and 126, and on the leg between element 125 and 127, respectively.

Steps 300 may be performed after detecting benchmark elements 124, 125, 126, 127 in step 102 in FIG. 1. In a first step 301, the depicted location values 313 of elements 124, 125, 126, 127 may be determined as depicted 340 in the digital image. In a following step 302, a depicted polygon 341 may be determined based on the depicted location values 313 of the respective benchmark elements 124, 125, 126, 127. In other words, the benchmark elements 124, 125, 126, 127 as depicted in the digital image may define a depicted polygon 341. For example, FIG. 3 illustrates calibration marker 320 as depicted in a digital image wherein the depicted calibration marker 340 is substantially skewed or warped. This can, for example, occur when the digital image was not captured at a substantially rectangular angle or substantially transverse to the skin tissue, thereby warping the perspective of the skin tissue and the calibration marker in the digital image.

In a next step 303, the perspective of the digital image may be adjusted based on the benchmark polygon 321 and the depicted polygon 341. This can be achieved by a geometric image transformation that warps the depicted polygon 341 to the shape of the benchmark polygon 321. In other words, the pixel grid of the digital image is deformed based on the deviation between the depicted polygon 341 and the benchmark polygon 321. The pixels within the depicted polygon 341 can thus be mapped to the pixels within the benchmark polygon 321 according to a geometric transformation matrix. In doing so, geometric distortions in the digital image can be substantially corrected thereby obtaining a perspective calibrated image 315.

Adjusting the perspective of the digital image allows assessing skin tissue more reliably based on a digital image regardless of factors that influence the perspective of a digital image, e.g. optical aberrations or camera angle. Correcting the perspective of the digital image further allows to determine the size of skin lesions more accurately. This has the advantage that the size and shape of skin lesions may be rendered substantially true within the perspective calibrated digital image 315, regardless of the conditions wherein the digital image is taken or which equipment has been used to capture the digital image. In other words, regardless of how the digital image was taken. This has the further advantage that the severity and/or evolution of a skin condition can be determined more reliably, as the shape and size of a skin lesion are typical visible signs for skin conditions.

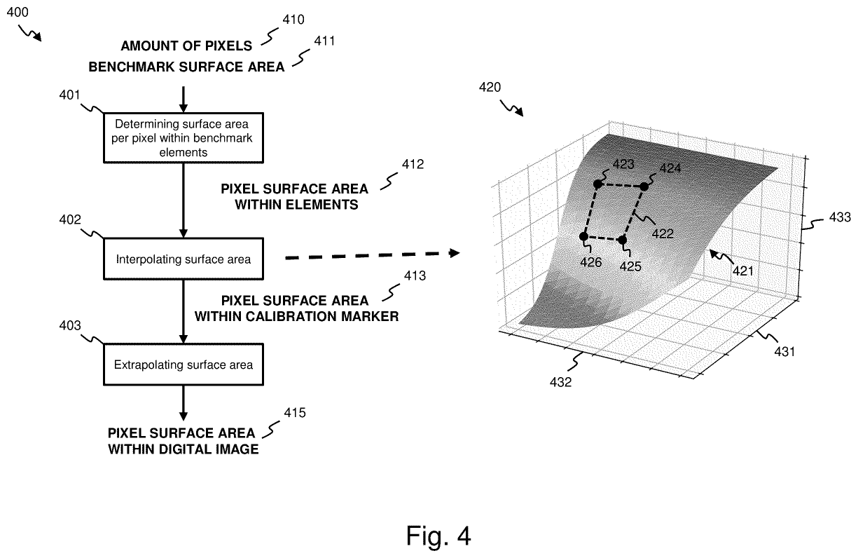

FIG. 4 shows steps 400 of the computer-implemented method further comprising determining a surface area associated with the respective pixels of the digital image, according to embodiments. To this end, one or more benchmark elements may be characterized by a benchmark surface area value of a surface area type. The one or more benchmark elements characterized by a surface area value may include benchmark elements 124, 125, 126, 127 of FIGS. 1 and 3 that are characterized by a location value. Alternatively or complementary, the one or more benchmark elements characterized by a surface area value may include benchmark elements 121, 122, 123 of FIGS. 1 and 2 that are characterized by a colour value.

Steps 400 may be performed after detecting the respective benchmark elements within the digital image in step 102 in FIG. 1. In a first step 401, a surface area associated with the respective pixels within the benchmark elements can be determined based on the amount of pixels 410 located within the benchmark elements and the benchmark surface area 411 of these benchmark elements. The amount of pixels 410 within each benchmark element can thus be determined. Thereafter, dividing the benchmark surface area by the determined amount of pixels 410 allows obtaining a surface area value for the respective pixels within the benchmark elements, i.e. a pixel surface area 412.

It will be apparent that the pixel surface area 412 can be different for respective benchmark elements, e.g. due to the perspective of the digital image. The surface area associated with pixels located outside the benchmark elements can thus also vary substantially due to the image perspective. Pixels located outside the benchmark elements may include pixels located within the unmarked body of the calibration marker or pixels not located on the calibration marker. Surface plot 420 illustrates an example of the surface area values 421 associated with the respective pixels within a digital image of skin tissue. Axis 433 may represent the magnitude of the surface area value; and axes 431, 432 may represent x-coordinates and y-coordinates of the pixel grid of the digital image, respectively. It will be apparent that a digital image of skin tissue wherein the pixels have surface area values according to surface plot 420 cannot be used to accurately determine the size or dimensions of skin lesions without determining the surface area values 421 for the respective pixels in the digital image.

To this end, the computer-implemented method may further comprise interpolating the surface area associated with the pixels located within the benchmark elements in step 402. For example, points 423, 424, 425, 426 may illustrate respective benchmark elements characterized by respective pixel surface areas 412. Interpolating between the respective benchmark elements 423, 424, 425, 426 allows obtaining a more accurate surface area value for the pixels located within the polygon 422 defined by the elements 423, 424, 425, 426, e.g. pixels located within the calibration marker. Elements 423, 424, 425, 426 may, for example, correspond to benchmark elements 124, 125, 126, 127 in FIGS. 1 and 3. Polygon 422 may, for example, correspond to the depicted polygon 341 in FIG. 3. This allows accurately determining the size or dimensions of skin lesions located between the benchmark elements, e.g. inside the boundaries of the calibration marker.

Alternatively or complementary, the computer-implemented method may further comprise extrapolating the surface area associated with the pixels located within the benchmark elements. This extrapolation may additionally be based on the interpolated surface area associated with the pixels located within polygon 422. Extrapolating the pixel surface area thus allows obtaining a more accurate surface area value for the pixels located outside polygon 422, e.g. pixels located outside the calibration marker. This allows accurately determining the size or dimensions of skin lesions not located between benchmark elements, e.g. outside the boundaries of the calibration marker.

It will be apparent that, additional benchmark elements arranged within polygon 422, e.g. between elements 423 and 426 and between elements 424 and 425, can further improve the accuracy of the interpolation and/or extrapolation. It will further be apparent that the interpolating in step 402 and the extrapolating in step 403 can be performed in any order, or substantially simultaneously.

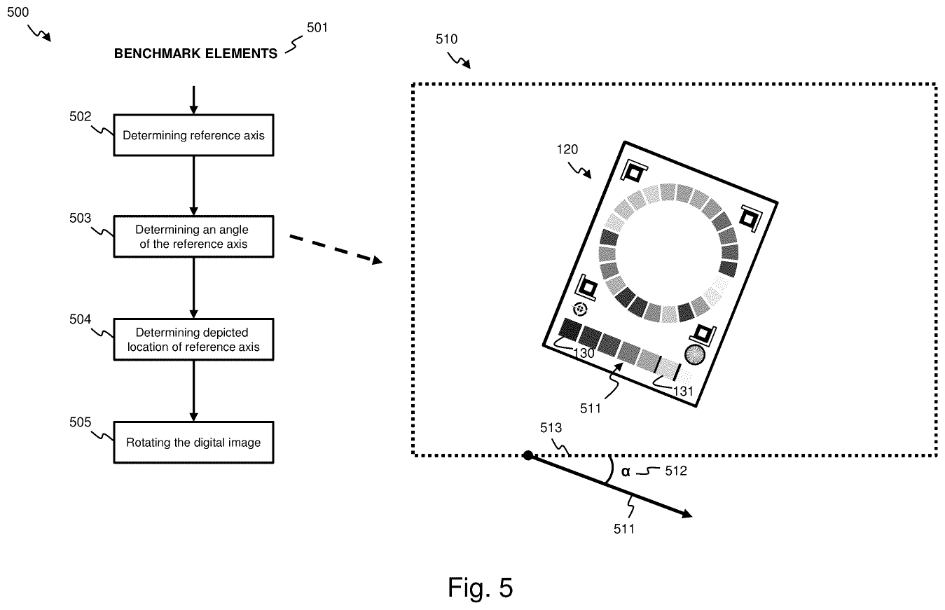

FIG. 5 shows steps 500 of the computer-implemented method for rotating the digital image based on a reference axis 511, according to embodiments. To this end, one or more benchmark elements define a reference axis 511 of the calibration marker 120. The calibration marker 120 may comprise a plurality of benchmark elements 130, 131 that are lined up, thereby defining the reference axis 511. The reference axis may, for example, be defined by respective predetermined pixels or points within the plurality of lined-up benchmark elements. For example, FIG. 5 illustrates seven benchmark elements 130, 131 that are lined up such that the geometric centre of the respective benchmark elements 130, 131 defines the references axis 511.

The plurality of benchmark elements 130, 131 may further be characterized by one or more additional attribute values of different attribute types, e.g. colour values. For example, the seven benchmark elements 130, 131 in FIG. 5 may be characterized by respective grey scale colour values. Alternatively, the calibration marker 120 may comprise a benchmark element substantially shaped as a line or an arrow indicative for the reference axis 511.

Steps 500 may be performed after detecting the benchmark elements within the digital image in step 102 in FIG. 1. In a first step 502, the reference axis 511 may be determined from the detected benchmark elements 501. In a next step 503, an angle 512 between the reference axis 512 and a predetermined edge 513 of the digital image 510 may be determined. The predetermined edge 513 can be any outer edge of the digital image 510. In a following step 505, the digital image may be rotated to a calibrated position based on the angle 512. The digital image 510 may be rotated such that the reference axis is oriented substantially horizontal. For example in FIG. 5, the digital image 510 may be rotated anti-clockwise by an angle corresponding to angle 512. Alternatively, the digital image may be rotated such that the reference axis is oriented substantially vertical. For example, when the reference axis 511 is located in the longitudinal direction of the calibration marker 120, i.e. transverse on the reference axis 511 in FIG. 5.

The reference axis 511, i.e. the benchmark elements 130, 131, may further be characterized by a benchmark location on the calibration marker 120. The reference axis 511 may be arranged on the surface of the calibration marker 120 such that the calibration marker is asymmetric. For example, reference axis 511 is arranged substantially towards the bottom outer limit of calibration marker 120. This allows orienting the calibration marker according to a predetermined orientation, e.g. such that the reference axis is positioned at the bottom of the calibration marker 120. This can avoid that the digital image 510 is rotated to an inverted orientation. To this end, the depicted location of the reference axis may be determined in step 504, and the digital image may be rotated based on this depicted location compared to the benchmark location of the reference axis in addition to the angle 512 in step 505.

The computer-implemented method may further comprise estimating the resolution of the digital image. To this end, the calibration marker may include a siemens star 129. If the resolution does not meet a minimum threshold, a user may be prompted to repeat the capturing of the digital image or to provide a digital image with a higher resolution. Estimating the resolution of the digital image can be performed at any time during the computer-implemented method, e.g. before detecting the calibration marker in step 101 in FIG. 1, or after calibrating the digital image in step 104 in FIG. 1. This can ensure that the resolution of the calibrated image 115 is sufficient to assess a skin condition based on the digital image.

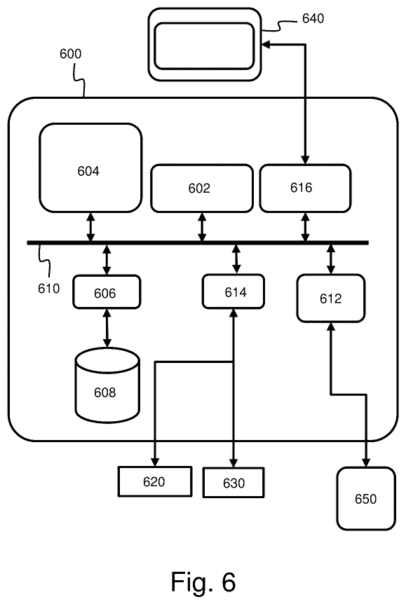

FIG. 6 shows a suitable computing system 600 enabling to implement embodiments of the above described method according to the invention. Computing system 600 may in general be formed as a suitable general-purpose computer and comprise a bus 610, a processor 602, a local memory 604, one or more optional input interfaces 614, one or more optional output interfaces 616, a communication interface 612, a storage element interface 606, and one or more storage elements 608. Bus 610 may comprise one or more conductors that permit communication among the components of the computing system 600. Processor 602 may include any type of conventional processor or microprocessor that interprets and executes programming instructions. Local memory 604 may include a random-access memory (RAM) or another type of dynamic storage device that stores information and instructions for execution by processor 602 and/or a read only memory (ROM) or another type of static storage device that stores static information and instructions for use by processor 602. Input interface 614 may comprise one or more conventional mechanisms that permit an operator or user to input information to the computing device 600, such as a keyboard 620, a mouse 630, a pen, voice recognition and/or biometric mechanisms, a camera, etc. Output interface 616 may comprise one or more conventional mechanisms that output information to the operator or user, such as a display 640, etc. Communication interface 612 may comprise any transceiver-like mechanism such as for example one or more Ethernet interfaces that enables computing system 600 to communicate with other devices and/or systems such as, for example, a smartphone 650. The communication interface 612 of computing system 600 may be connected to such another computing system by means of a local area network (LAN) or a wide area network (WAN) such as for example the internet. Storage element interface 606 may comprise a storage interface such as for example a Serial Advanced Technology Attachment (SATA) interface or a Small Computer System Interface (SCSI) for connecting bus 610 to one or more storage elements 608, such as one or more local disks, for example SATA disk drives, and control the reading and writing of data to and/or from these storage elements 608. Although the storage element(s) 608 above is/are described as a local disk, in general any other suitable computer-readable media such as a removable magnetic disk, optical storage media such as a CD or DVD, -ROM disk, solid state drives, flash memory cards, etc. could be used.

Although the present invention has been illustrated by reference to specific embodiments, it will be apparent to those skilled in the art that the invention is not limited to the details of the foregoing illustrative embodiments, and that the present invention may be embodied with various changes and modifications without departing from the scope thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein. In other words, it is contemplated to cover any and all modifications, variations or equivalents that fall within the scope of the basic underlying principles and whose essential attributes are claimed in this patent application. It will furthermore be understood by the reader of this patent application that the words “comprising” or “comprise” do not exclude other elements or steps, that the words “a” or “an” do not exclude a plurality, and that a single element, such as a computer system, a processor, or another integrated unit may fulfil the functions of several means recited in the claims. Any reference signs in the claims shall not be construed as limiting the respective claims concerned. The terms “first”, “second”, third”, “a”, “b”, “c”, and the like, when used in the description or in the claims are introduced to distinguish between similar elements or steps and are not necessarily describing a sequential or chronological order. Similarly, the terms “top”, “bottom”, “over”, “under”, and the like are introduced for descriptive purposes and not necessarily to denote relative positions. It is to be understood that the terms so used are interchangeable under appropriate circumstances and embodiments of the invention are capable of operating according to the present invention in other sequences, or in orientations different from the one(s) described or illustrated above.

1.-15. (canceled)

16. A computer-implemented method for calibrating a digital image of skin tissue,
wherein the digital image comprises a calibration marker located on or near the skin tissue, and
wherein the calibration marker comprises benchmark elements wherein at least one benchmark attribute value of at least one attribute type; the computer-implemented method comprising:
detecting the calibration marker within the digital image;
detecting the benchmark elements within a cropped portion of the digital image that includes the calibration marker;
for the respective benchmark elements, determining at least one depicted attribute value of the at least one attribute type based on pixels of the digital image located within the respective benchmark elements; and
calibrating the digital image by correcting deviations between the depicted attribute values and the benchmark attribute values associated with the respective benchmark elements.

17. The computer-implemented method according to claim 16, wherein detecting the calibration marker is performed by a machine learning model trained for detecting the calibration marker within a digital image.

18. The computer-implemented method according to claim 16, wherein detecting the benchmark elements is performed by a machine learning model trained for assigning respective pixels within the cropped portion of the digital image to the benchmark elements.

19. The computer-implemented method according to claim 16, wherein one or more benchmark elements are wherein respective benchmark color values of a color type; and
wherein calibrating the digital image comprises correcting color values of pixels within the digital image.

20. The computer-implemented method according to claim 19, wherein determining at least one depicted attribute value of the color type comprises determining an average depicted color value of the pixels of the digital image located within the respective one or more benchmark elements.

21. The computer-implemented method according to claim 20, further comprising determining respective color deviations between the average depicted color value and the benchmark color value associated with the respective one or more benchmark elements.

22. The computer-implemented method according to claim 16, wherein at least four benchmark elements are characterized by a benchmark location value of a location type; and
wherein calibrating the digital image comprises adjusting a perspective of the digital image.

23. The computer-implemented method according to claim 22, wherein determining at least one depicted attribute value of the location type comprises determining a depicted location of the respective at least four benchmark elements.

24. The computer-implemented method according to claim 23, further comprising determining a depicted polygon defined by the depicted locations of the respective at least four benchmark elements within the digital image; and
wherein adjusting the perspective of the digital image further comprises mapping pixels within the depicted polygon to pixels within a benchmark polygon defined by the benchmark location value of the at least four benchmark elements.

25. The computer-implemented method according to claim 16, wherein one or more benchmark elements define a reference axis of the calibration marker, and
wherein calibrating the digital image comprises rotating the digital image based on the reference axis.

26. The computer-implemented method according to claim 25, further comprising determining an angle between the reference axis of the calibration marker and a predetermined edge of the digital image.

27. The computer-implemented method according to claim 25, wherein the reference axis is characterized by a benchmark location on the calibration marker; and
wherein rotating the digital image is further based on a depicted location of the reference axis.

28. The computer-implemented method according to claim 16, wherein one or more benchmark elements are characterized by a benchmark surface area value of a surface area type; the computer-implemented method further comprising determining a surface area associated with the respective pixels of the digital image based on an amount of pixels located within the respective one or more benchmark elements and the benchmark surface area of the respective one or more benchmark elements.

29. The computer-implemented method according to claim 27, wherein determining the surface area associated with pixels located outside the one or more benchmark elements comprises interpolating and/or extrapolating the surface area associated with the pixels located within the one or more benchmark elements.

30. A data processing system configured to perform the computer-implemented method according to claim 16.
